(19) United States
(12) Patent Application Publication
(10) Pub. No.: US 2021/0178186 A1
(43) Pub. Date: Jun. 17, 2021

(54) FOCUSING HEAD AND RADIOTHERAPY EQUIPMENT

(71) Applicants: OUR UNITED CORPORATION, Xi'an City, Shaanxi (CN); SHENZHEN OUR NEW MEDICAL TECHNOLOGIES DEVELOPMENT CO., LTD., Shenzhen City, Guangdong (CN)

(72) Inventors: Fangzheng CHEN, Xi'an City, Shaanxi (CN); Hua YANG, Xi'an City, Shaanxi (CN)

(21) Appl. No.: 17/256,967

(22) PCT Filed: Jun. 25, 2019
(86) PCT No.: PCT/CN2019/092802

§ 371 Date: Dec. 29, 2020

(30) Foreign Application Priority Data
Jun. 29, 2018 (CN) 201821046013.2

(51) Int. Cl. A61N 5/10

(52) U.S. Cl. CPC A61N 5/1007; A61N 2005/101; A61N 2005/1094

(57) ABSTRACT

Provided is a focusing head, including: a cartridge configured to carry a plurality of radiation sources; a shielding roller with the cartridge therein; a first driving assembly connected to the cartridge and configured to drive the cartridge to rotate; and a second driving assembly connected to the shielding roller and configured to drive the shielding roller to rotate.

The present disclosure is a US national phase application of PCT Application No. PCT/CN2019/092802, filed on Jun. 25, 2019, which claims priority to Chinese Patent Application No. 201821046013.2, filed on Jun. 29, 2018 and entitled “FOCUSING HEAD AND RADIOTHERAPY APPARATUS,” the entire contents of each of which are incorporated herein by reference.

### TECHNICAL FIELD

The present disclosure relates to the field of medical technologies, in particular relates to a focusing head and radiotherapy equipment.

### BACKGROUND

A gamma knife is large-scale medical equipment mainly used for treating craniocerebral diseases. Based on the three-dimensional geometric orientation, the gamma knife selectively determines normal tissue or diseased intracranial tissue as a target, and then focuses gamma rays generated by cobalt-60 on the target, the gamma rays irradiate the target in a large dose at one time, such that the targets are subject to focal necrosis or functional changes, thereby achieving the purpose of treating the diseases.

### SUMMARY

The present disclosure provides a focusing head and radiotherapy equipment. The technical solutions are described as below.

In a first aspect, a focusing head is provided. The focusing head includes:

a cartridge configured to carry a plurality of radiation sources;

a shielding roller with the cartridge therein;

a first driving assembly connected to the cartridge and configured to drive the cartridge to rotate; and

a second driving assembly connected to the shielding roller and configured to drive the shielding roller to rotate.

Optionally, the first driving assembly includes a first driving motor, and a first driving structure connected to the cartridge, wherein the first driving motor is configured to drive the cartridge to rotate via the first driving structure.

The second driving assembly includes a second driving motor, and a second driving structure connected to the shielding roller, wherein the second driving motor is configured to drive the shielding roller to rotate via the second driving structure.

Optionally, the first driving structure includes a cartridge driving gear set.

The cartridge driving gear set includes a first motor driving gear connected to the first driving motor, and a cartridge driving gear connected to the cartridge.

Optionally, the cartridge driving gear set further includes a first intermediate gear, wherein the first intermediate gear is engaged with both the first motor driving gear and the cartridge driving gear.

Optionally, the second driving structure includes a roller driving gear set.

The roller driving gear set includes a second motor driving gear connected to the second driving motor, and a roller driving gear connected to the shielding roller.

Optionally, the roller driving gear set further includes a second intermediate gear, wherein the second intermediate gear is engaged with both the second motor driving gear and the roller driving gear.

Optionally, an outer surface of the cartridge is tangent to an inner surface of the shielding roller, and the plurality of radiation sources are disposed on the side, tangent to the shielding roller, in the cartridge.

Optionally, the shielding roller includes a roller body, and a plurality of ray channels disposed on a side wall of the roller body;

wherein the plurality of radiation sources are communicated with the plurality of ray channels in one-to-one correspondence when the focusing head is in a source-on state, and the plurality of radiation sources are staggered with the plurality of ray channels when the focusing head is in a source-off state.

Optionally, the first driving assembly is configured to drive the cartridge to rotate when the focusing head is switched between the source-on state and the source-off state.

Optionally, the second driving assembly is configured to drive the shielding roller to rotate when the focusing head is switched between the source-on state and the source-off state.

Optionally, when the focusing head is switched between the source-on state and the source-off state, the first driving assembly is configured to drive the cartridge to rotate in a first direction and the second driving assembly is configured to drive the shielding roller to rotate in a second direction, wherein the first direction is opposite to the second direction.

Optionally, the cartridge and the shielding roller rotate relative to each other at an acute angle.

Optionally, the first driving assembly is configured to drive the cartridge to rotate by 180°, and the second driving assembly is configured to drive the shielding roller to rotate by 90°.

Optionally, an external diameter of the cartridge is less than an internal diameter of the shielding roller.

Optionally, the cartridge is driven by the first driving assembly to rotate around a center axis of the shielding roller, and the shielding roller is driven by the second driving assembly to rotate around the center axis of the shielding roller.

Optionally, a distance between a center axis of the cartridge and the center axis of the shielding roller is 22 mm.

Optionally, the focusing head further includes a shielding layer for cladding the shielding roller.

Optionally, the focusing head further includes a collimator with a plurality of collimation channels thereon, wherein the plurality of ray channels are communicated with the plurality of collimation channels in one-to-one correspondence when the focusing head is in the source-on state.

Optionally, a material of the shielding roller includes a tungsten alloy.

In a second aspect, radiotherapy equipment is provided. The radiotherapy equipment includes a rotating gantry and a focusing head connected to the rotating gantry. The focusing head includes:

a cartridge configured to carry a plurality of radiation sources;

a shielding roller with the cartridge therein;

a first driving assembly connected to the cartridge and configured to drive the cartridge to rotate; and

a second driving assembly connected to the shielding roller and configured to drive the shielding roller to rotate.

### BRIEF DESCRIPTION OF THE DRAWINGS

For clearer descriptions of the technical solutions in the embodiments of the present disclosure, the following briefly introduces the accompanying drawings required for describing the embodiments. Apparently, the accompanying drawings as described below show merely some embodiments of the present disclosure, and a person of ordinary skill in the art may also derive other drawings from these accompanying drawings without creative efforts.

FIG. 1 is a schematic structural diagram of a focusing head in the related art;

FIG. 2 is a schematic diagram of the focusing head of FIG. 1 in a source-on state;

FIG. 3 is a schematic diagram of the focusing head of FIG. 1 in a source-off state;

FIG. 4 is a schematic diagram of the focusing head of FIG. 1 of which the sources are quickly turned off;

FIG. 5 is a schematic structural diagram of a focusing head according to an embodiment of the present disclosure;

FIG. 6 is a side view of the focusing head shown in FIG. 5;

FIG. 7 is a structural block diagram of a focusing head according to an embodiment of the present disclosure;

FIG. 8 is a schematic structural diagram of another focusing head according to an embodiment of the present disclosure;

FIG. 9 is a schematic structural diagram of yet another focusing head according to an embodiment of the present disclosure;

FIG. 10 is a schematic structural diagram of still another focusing head according to an embodiment of the present disclosure;

FIG. 11 is a schematic diagram of the focusing head of FIG. 9 in a source-on state;

FIG. 12 is a schematic diagram of the focusing head of FIG. 9 in a source-off state; and

FIG. 13 is a schematic diagram of the focusing head in FIG. 9 after the sources are quickly turned off.

### DETAILED DESCRIPTION

To present the object, technical solutions and advantages of the present disclosure more clearly, the embodiments of the present disclosure are described in further detail with reference to the accompanying drawings.

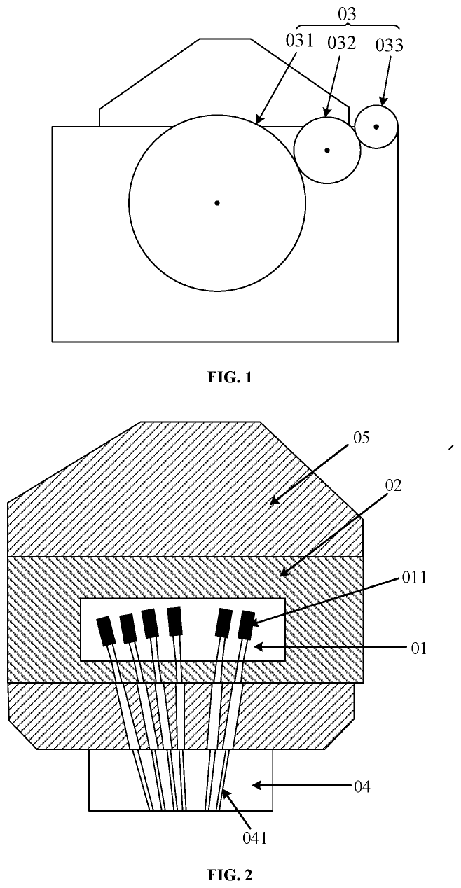

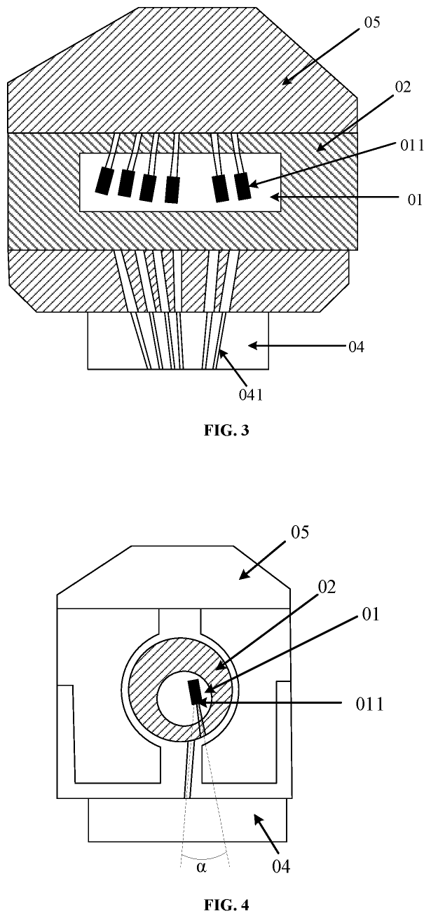

In the related art, referring to FIG. 1, FIG. 2, and FIG. 3, as a main component of the gamma knife, a focusing head may include a cobalt source cartridge 01, a tungsten roller 02, a tungsten roller driving assembly 03, a collimator 04, a shield 05, and the like. A plurality of radiation sources 011 are disposed in the cobalt source cartridge 01.

The cobalt source cartridge 01 is disposed in and connected to the tungsten roller 02, such that the cobalt source cartridge 01 and the tungsten roller 02 is capable of moving synchronously.

The tungsten roller driving assembly 03 includes a tungsten roller driving gear 031, a intermediate gear 032, a motor driving gear 033, and a driving motor (not shown). The tungsten roller driving gear 031, the intermediate gear 032, and the motor driving gear 033 are sequentially engaged with one another. The tungsten roller driving gear 031 is connected to the tungsten roller 02, and the motor driving gear 033 is connected to the driving motor. The driving motor drives the motor driving gear 033 to rotate and thereby drives the intermediate gear 032 and the tungsten roller driving gear 031 to rotate, and the tungsten roller driving gear 031 drives the tungsten roller 02 to rotate. That is, the driving motor drives the tungsten roller 02 and the cobalt source cartridge 01 in the tungsten roller 02 to rotate.

Generally, the focusing head may be in a source-on state or a source-off state. As shown in FIG. 2, when the focusing head is in a source-on state, rays emitted by the plurality of radiation sources 011 in the cobalt source cartridge 01 need to come out as beams after passing through a plurality of collimation holes 041 in the collimator 04.

As shown in FIG. 3, when the focusing head is in a source-off state, rays emitted by the plurality of radiation sources 011 in the cobalt source cartridge 01 need to be shielded by the shield 05.

When the focusing head is switched between a source-on state and a source-off state, the driving motor needs to simultaneously drive the tungsten roller and the cobalt source cartridge disposed in the tungsten roller to rotate. In this way, a driving operation load is high, leading to a longer time period required for switching the focusing head between the source-on state and the source-off state, and further resulting in a low therapeutic efficiency of the focusing head.

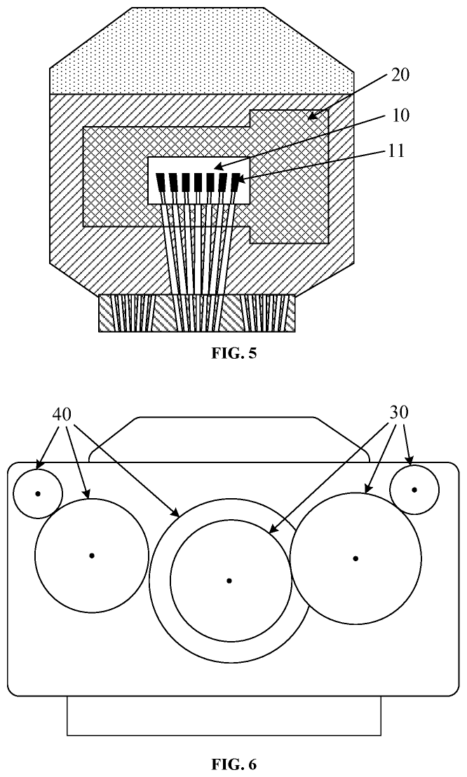

As shown in FIG. 4, when the focusing head needs to be quickly switched from the source-on state to the source-off state (that is, the sources of the focusing head need to be quickly turned off), the driving motor needs to drive the tungsten roller 02 and the cobalt source cartridge 01 disposed in the tungsten roller 02 to rotate at a specified angle α, such that the shield 05 disposed outside the tungsten roller 01 shields the rays emitted by the radiation sources 011. Since the tungsten roller and the cobalt source cartridge disposed in the tungsten roller, which are required to be driven by the driving motor, are relatively heavy, it takes a long time for the driving motor to drive the tungsten roller and the cobalt source cartridge disposed in the tungsten roller to rotate to a specified angle.

Referring to FIGS. 5 and 6, FIG. 5 is a schematic structural diagram of a focusing head according to an embodiment of the present disclosure, and FIG. 6 is a side view of the focusing head shown in FIG. 5.

The focusing head includes: a cartridge 10, a shielding roller 20, a first driving assembly 30, and a second driving assembly 40.

The cartridge 10 is configured to carry a plurality of radiation sources 11 that may be cobalt-60 radiation sources. The cartridge 10 is disposed in the shielding roller 20.

The cartridge 10 is connected to the first driving assembly 30, and the first driving assembly 30 is configured to drive the cartridge 10 to rotate. The shielding roller 20 is connected to the second driving assembly 40, and the second driving assembly 40 is configured to drive the shielding roller 20 to rotate.

Compared with the traditional solution that a focusing head simultaneously drives a cobalt source cartridge and a tungsten roller to rotate by one driving assembly, the focusing head in this embodiment drives the cartridge 10 and the shielding roller 20 to rotate via the first driving assembly 30 and the second driving assembly 40 respectively. In this way, the driving operation load of each driving assembly is reduced, and thus a time period required for switching the focusing head between the source-on state and the source-off state is shortened, thereby improving the therapeutic efficiency of the focusing head.

Currently, international standards require that the sources of the focusing head to be quickly turned off within 0.2 seconds. In the related art, since a driving motor needs to simultaneously drive the tungsten roller and the cobalt source cartridge in the tungsten roller to rotate and the driving operation load is high accordingly, it is difficult to quickly turn off the sources of the focusing head within 0.2 seconds. In this embodiment, since the first driving assembly 30 and the second driving assembly 40 respectively drive the cartridge 10 and the shielding roller 20 to rotate, and the driving operation load of each driving assembly is low, thus the time required for quickly turning off the sources of the focusing head can be shortened. In this embodiment, it is easier to quickly turn off the sources of the focusing head within 0.2 seconds, such that the therapeutic efficiency of the focusing head is further improved.

In summary, according to the focusing head in this embodiment, the first driving assembly is connected to the cartridge and configured to drive the cartridge to rotate, and the second driving assembly is connected to the shielding roller and configured to drive the shielding roller to rotate. In this way, the driving operation load of each driving assembly is effectively reduced, and thus a time period required for switching the focusing head between the source-on state and the source-off state is shortened, thereby effectively improving the therapeutic efficiency of the focusing head. In addition, time required for quickly turning off the sources of the focusing head is shortened, such that the therapeutic efficiency of the focusing head is further improved.

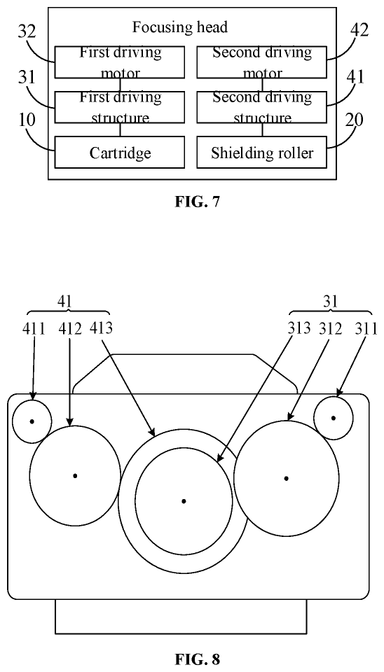

Optionally, referring to FIG. 7, a structural block diagram of a focusing head according to an embodiment of the present disclosure is shown. The first driving assembly in the focusing head includes a first driving motor 32, and a first driving structure 31 connected to the cartridge 10. The first driving motor 32 is configured to drive the cartridge 10 to rotate via the first driving structure 31. The second driving assembly in the focusing head includes a second driving motor 42, and a second driving structure 41 connected to the shielding roller 20. The second driving motor 42 is configured to drive the shielding roller 20 to rotate via the second driving structure 41.

It should be noted that the first driving structure 32 and the second driving structure 42 may be the same or different. For example, both the first driving structure 32 and the second driving structure 42 are gear sets; or both of them are coordinated structures of gear and rack; or one of the first driving structure 32 and the second driving structure 42 is a gear set, and the other is a coordinated structure of gear and rack. The following embodiments take a case where both the first driving structure 32 and the second driving structure 42 are gear sets as an example for illustrative descriptions.

Illustratively, referring to FIG. 7 and FIG. 8. FIG. 8 is a schematic structural diagram of another focusing head according to an embodiment of the present disclosure. The first driving structure 31 in the first driving assembly 30 includes a cartridge driving gear set. The cartridge driving gear set includes a first motor driving gear 311 and a cartridge driving gear 313. The first motor driving gear 311 is connected to the first driving motor 32, and the cartridge driving gear 313 is connected to the cartridge 10. Optionally, the cartridge driving gear set further includes a first intermediate gear 312. The first intermediate gear 312 is engaged with both the first motor driving gear 311 and the cartridge driving gear 313. The first driving motor 32 drives the first motor driving gear 311 to rotate, and thereof to drive the first intermediate gear 312 and the cartridge driving gear 313 to rotate, and thus the cartridge driving gear 313 drive the cartridge 10 to rotate, such that the first driving motor 32 drives the cartridge 10 to rotate.

The second driving structure 41 in the second driving assembly 40 includes a roller driving gear set. The roller driving gear set includes a second motor driving gear 411 and a roller driving gear 413. The second motor driving gear 411 is connected to the second driving motor 42, and the roller driving gear 413 is connected to the shielding roller 20. Optionally, the roller driving gear set further includes a second intermediate gear 412. The second intermediate gear 412 is engaged with both the second motor driving gear 411 and the roller driving gear 413. The second driving motor 42 drives the second motor driving gear 411 to rotate and thereof to drive the second intermediate gear 412 and the roller driving gear 413 to rotate, and thus the roller driving gear 413 drives the shielding roller 20 to rotate, such that the second driving motor 42 drives the shielding roller 20 to rotate.

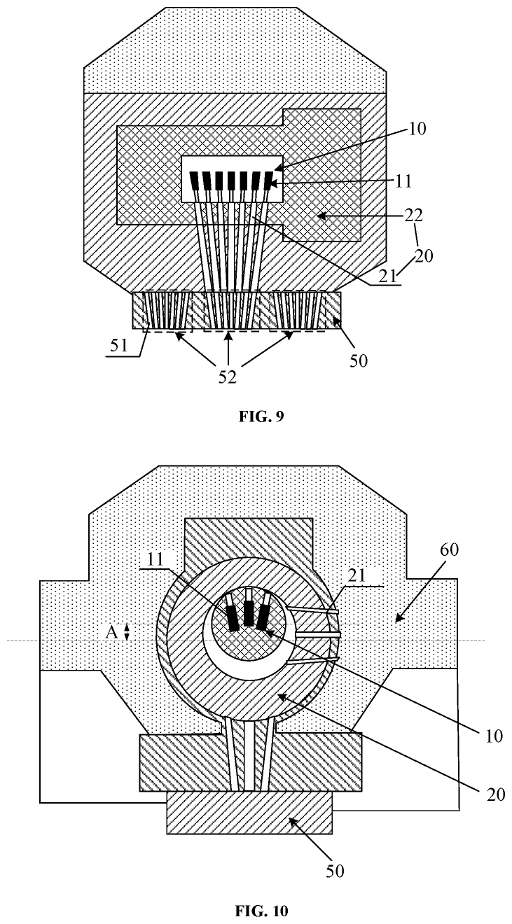

In this embodiment, referring to FIG. 9, a schematic structural diagram of yet another focusing head according to an embodiment of the present disclosure is shown. A plurality of radiation sources 11 are carried in the cartridge 10, and the shielding roller 20 includes a roller body 22, and a plurality of ray channels 21 disposed on a side wall of the roller body 22. It should be noted that in this embodiment, an area, where the ray channels 21 are not disposed, on the roller body 22 in the shielding roller 20 is a shielding area. The focusing head further includes a collimator 50, and a plurality of collimation channels 51 are disposed on the collimator 50. It also should be noted that the plurality of collimation channels 51 may be divided into a plurality of collimation hole groups 52, and the number of the collimation channels 51 in each collimation hole group 52 equals the number of the radiation sources 11 carried in the cartridge 10.

In in an example embodiment, referring to FIG. 10, FIG. 10 is a schematic structural diagram of still another focusing head according to an embodiment of the present disclosure. In order to ensure that the shielding area in the shielding roller 20 shields rays emitted by the radiation sources 11, an outer surface of the cartridge 10 needs to be tangent to an inner surface of the shielding roller 20, and the plurality of radiation sources 11 in the cartridge 10 are disposed on the side, tangent to the shielding roller 20, of the cartridge 10. At this time, the rays emitted by the radiation sources 11 is directly shielded by the side, tangent to the cartridge, of the shielding roller 20.

Optionally, an external diameter of the cartridge 10 is less than an internal diameter of the shielding roller 20. The cartridge 10 is driven by the first driving assembly to rotate around a center axis of the shielding roller 20, and the shielding roller 20 is driven by the second driving assembly to rotate around the center axis of the shielding roller 20. That is, a rotation axis of the cartridge 10 driven to rotate by the first driving assembly is coincided with a rotation axis of the shielding roller 20 driven to rotate by the second driving assembly. At this time, a center axis of the cartridge 10 is not coincided with the rotation axis of the cartridge 10, an offset A is present between the center axis of the cartridge 10 and the rotation axis of the cartridge 10, wherein the offset A may be 22 mm. That is, the distance between the center axis of the cartridge 10 and the center axis of the shielding roller is 22 mm.

Optionally, the focusing head further includes a shielding layer 60 for cladding the shielding roller 20. International standards require that a radiation dose measured 5 cm away from the surface of the focusing head should not be greater than 200 uSv/h (micro sievert per hour). If the radiation sources 11 are cobalt-60 radiation sources, and a material of the shielding roller 20 includes a tungsten alloy and a material of the shielding layer includes a steel-lead alloy, the total thickness of the shielding roller 20 and the shielding layer 60 may not be less than 180 mm.

In the embodiments of the present disclosure, the cartridge 10 is driven by the first driving assembly 30, and/or the shielding roller 20 is driven by the second driving assembly 40, such that the focusing head is in the source-on state or the source-off state.

Referring to FIG. 11, FIG. 11 is a schematic diagram of the focusing head of FIG. 9 in a source-on state. When the plurality of radiation sources 10 are communicated with the plurality of ray channels 21 in one-to-one correspondence, and the plurality of ray channels 21 are communicated with the plurality of collimation channels 51 in one collimation hole group 52 on the collimator 50 in one-to-one correspondence, the focusing head is in the source-on state. At this time, the rays emitted by the plurality of radiation sources 11 in the cartridge 10 come out as beams after sequentially passing through the corresponding ray channels 21 in the shielding roller 20 and the corresponding collimation channels 51 in one collimation hole group 52 in the collimator 50.

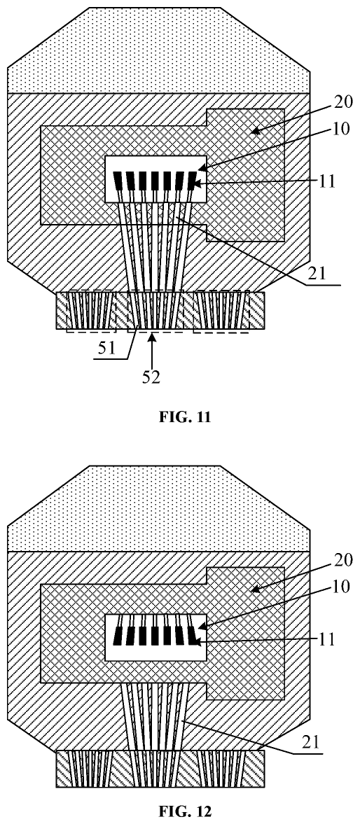

Referring to FIG. 12, FIG. 12 is a schematic diagram of the focusing head of FIG. 9 in a source-off state. When the plurality of radiation sources 10 are staggered with the plurality of ray channels 21, the focusing head is in the source-off state. At this time, the rays emitted by the plurality of radiation sources 11 in the cartridge 10 are shielded by the shielding area of the shielding roller 20.

In this embodiment, the focusing head may be switched between the source-on state and the source-off state in a plurality of ways. This embodiment takes the following three ways as examples for illustrative descriptions.

In a first way, the first driving assembly 30 is configured to drive the cartridge 10 to rotate, such that the focusing head may be switched between the source-on state and the source-off state. At this time, the second driving assembly 40 may not work, i.e., the shielding roller 20 keeps stationary. Illustratively, the first driving assembly 30 drives the cartridge 10 to rotate, such that the focusing head is switched from the source-on state shown in FIG. 11 to the source-off state shown in FIG. 12 or the focusing head is switched from the source-off state shown in FIG. 12 to the source-on state shown in FIG. 11.

In a second way, the second driving assembly 40 is configured to drive the shielding roller 20 to rotate, such that the focusing head is switched between the source-on state and the source-off state. At this time, the first driving assembly 30 may not work, i.e., the cartridge 10 keeps stationary. Illustratively, the second driving assembly 40 drives the shielding roller 20 to rotate, such that the focusing head is switched from the source-on state shown in FIG. 11 to the source-off state shown in FIG. 12, or, such that the focusing head is switched from the source-off state shown in FIG. 12 to the source-on state shown in FIG. 11.

In a third way, the first driving assembly 30 is configured to drive the cartridge 10 to rotate in a first direction, and the second driving assembly 40 is configured to drive the shielding roller 20 to rotate in a second direction, such that the focusing head is switched between the source-on state and the source-off state. The first direction is opposite to the second direction. Illustratively, the first driving assembly 30 drives the cartridge 10 to rotate and the second driving assembly 40 drives the shielding roller 20 to rotate, such that the focusing head is switched from the source-on state shown in FIG. 11 to the source-off state shown in FIG. 12 or the focusing head is switched from the source-off state shown in FIG. 12 to the source-on state shown in FIG. 11.

It should be noted that in another way, the first direction and the second direction may be the same, but it is necessary to ensure that an angle at which the first driving assembly 30 drives the cartridge 10 to rotate is different from an angle at which the second driving assembly 40 drives the shielding roller 20 to rotate.

Illustratively, when the focusing head is switched from the source-on state to the source-off state, as shown in FIG. 10, the first driving assembly drives the cartridge 10 to rotate in the first direction, such that the rays emitted by the plurality of radiation sources 11 in the cartridge 10 are shielded by the shielding area in the shielding roller 20; and the second driving assembly drives the shielding roller 20 to rotate in the second direction, such that the plurality of ray channels 21 in the shielding roller 20 are shielded by the shielding layer 60, thereby preventing the rays emitted by the radiation sources 11 in the cartridge 10 from passing through the plurality of ray channels 21. In this embodiment, the angle at which the first driving assembly drives the cartridge to rotate may be different from the angle at which the second driving assembly drives the shielding roller to rotate. For example, the first driving assembly may drive the cartridge to rotate by 180 degrees, and the second driving assembly may drive the shielding roller to rotate by 90 degrees.

In this embodiment, since the rays emitted by the radiation sources are shielded by the shielding area of the shielding roller 20 when the focusing head is in the source-off state, the thickness of the shielding layer in the focusing head can be reduced, thereby further effectively reducing the size of the focusing head.

In this embodiment, when the focusing head needs to be quickly switched from the source-on state to the source-off state (i.e., the sources of the focusing head need to be quickly turned off), the sources of the focusing head may also be quickly turned off by the aforementioned three implementation modes. Normally, when the sources of the focusing head are quickly turned off, it is necessary for the focusing head to ensure that the cartridge 10 and the shielding roller 20 rotate relative to each other at an angle, usually an acute angle, within 0.2 seconds, such that the focusing head is switched from the source-on state to the source-off state.

In the related art, when the sources of the focusing head need to be quickly turned off, as shown in FIG. 4, the driving motor needs to simultaneously drive the tungsten roller 02 and the cobalt source cartridge 01 in the tungsten roller 02 to rotate by a specified angle α. The rays emitted by the radiation sources 011 in the cobalt source cartridge 01 are shielded by the shield 05 disposed at the periphery of the tungsten roller 02 and the shielding area in the collimator 04. If the radiation sources 011 are cobalt-60 radiation sources, in order to enable the shield 05 and the shielding area in the collimator 04 to better shield the rays emitted by the radiation sources 011, it is necessary to ensure that the total thickness of the shield 05 and the shielding area in the collimator 04 is not less than 65 mm. Therefore, the driving motor needs to drive the tungsten roller 02 to rotate at the specified angle α, which has a certain requirement, and usually, the specified angle α is relatively great.

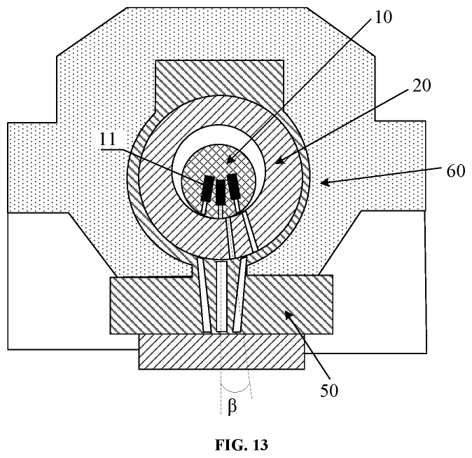

In this embodiment, referring to FIG. 13, a schematic diagram of the focusing head in FIG. 9 after the sources are quickly closed is shown. After the sources of the focusing head are quickly turned off under driving of the first driving assembly and/or the second driving assembly, the plurality of radiation sources 11 in the cartridge 10 are shielded by the shielding area in the shielding roller 20. If the radiation sources 11 in the cartridge 10 are cobalt-60 radiation sources, and a material of the shielding roller 20 includes a tungsten alloy, the thickness of the shielding roller 20 needs to be 65 mm in order to enable the shielding area in the shielding roller 20 to better shield the rays emitted by the radiation sources 11. After the sources of the focusing head are quickly turned off, the shielding area in the shielding roller 20 can directly shield the rays emitted by the radiation sources 11. Therefore, it is only necessary to ensure that the direction in which the radiation sources 11 emit the rays is staggered with an extending direction of the corresponding ray channel in the shielding roller 20. In this way, the direction in which each radiation source 11 emits the rays forms a relatively small specified angle β with the extending direction of the corresponding ray channel 21. That is, when the sources of the focusing head are quickly turned off, the angle β at which the cartridge 10 and the shielding roller rotate relative to each other, which further shortens time required for quickly turning off the sources of the focusing head.

Moreover, since the shielding area in the shielding roller 20 may directly shield the rays emitted by the radiation sources 11, neither the shielding layer 60 nor the shielding area on the collimator 50 needs to perform shielding, such that the collimator 50 no longer participates in the quick source-off process of the focusing head. The collimator 50 can be provided with more collimation hole groups, thereby effectively widening the treatment range of the focusing head.

In summary, according to the focusing head provided by this embodiment, the first driving assembly is connected to the cartridge and configured to drive the cartridge to rotate, and the second driving assembly is connected to the shielding roller and configured to drive the shielding roller to rotate. In this way, the driving operation load of each driving assembly is effectively reduced, and thus a time period required for switching the focusing head between the source-on state and the source-off state is shortened, thereby effectively improving the therapeutic efficiency of the focusing head. In addition, time required for quickly turning off the sources of the focusing head is shortened, such that the therapeutic efficiency of the focusing head is further improved. Meanwhile, since the rays emitted by the radiation sources are shielded by the shielding area of the shielding roller when the focusing head is in the source-off state, the thickness of the shielding layer in the focusing head can be reduced, making the size of the focusing head smaller. The collimator in the focusing head does not participate in the quick source-off process of the focusing head, such that the collimator can be provided with more collimation hole groups, thereby effectively widening the treatment range of the focusing head.

An embodiment of the present disclosure further provides a radiotherapy device. The radiotherapy device includes a rotating gantry and a focusing head connected to the rotating gantry. The focusing head may include a cartridge, a shielding roller, a first driving assembly and a second driving assembly. The cartridge is configured to carry a plurality of radiation sources and is disposed in the shielding roller. The first driving assembly is connected to the cartridge and configured to drive the cartridge to rotate. The second driving assembly is connected to the shielding roller and configured to drive the shielding roller to rotate. Illustratively, the focusing head is the focusing head shown in FIG. 5, FIG. 7, FIG. 8, FIG. 9 or FIG. 10.

Described above are merely example embodiments of the present disclosure, and are not intended to limit the present disclosure. Within the spirit and principles of the present disclosure, any modifications, equivalent substitutions, improvements, and the like are within the protection scope of the present disclosure.

1. A focusing head, comprising:
a cartridge configured to carry a plurality of radiation sources;
a shielding roller with the cartridge therein;
a first driving assembly connected to the cartridge and configured to drive the cartridge to rotate; and
a second driving assembly connected to the shielding roller and configured to drive the shielding roller to rotate.

2. The focusing head according to claim 1, wherein:
the first driving assembly comprises a first driving motor and a first driving structure connected to the cartridge, wherein the first driving motor is configured to drive the cartridge to rotate via the first driving structure; and
the second driving assembly comprises a second driving motor and a second driving structure connected to the shielding roller, wherein the second driving motor is configured to drive the shielding roller to rotate via the second driving structure.

3. The focusing head according to claim 2, wherein the first driving structure comprises a cartridge driving gear set; and
wherein the cartridge driving gear set comprises a first motor driving gear connected to the first driving motor and a cartridge driving gear connected to the cartridge.

4. The focusing head according to claim 3, wherein the cartridge driving gear set further comprises a first intermediate gear, wherein the first intermediate gear is engaged with both the first motor driving gear and the cartridge driving gear.

5. The focusing head according to claim 2, wherein the second driving structure comprises a roller driving gear set; and
wherein the roller driving gear set comprises a second motor driving gear connected to the second driving motor, and a roller driving gear connected to the shielding roller.

6. The focusing head according to claim 5, wherein the roller driving gear set further comprises a second intermediate gear, wherein the second intermediate gear is engaged with both the second motor driving gear and the roller driving gear.

7. The focusing head according to claim 1, wherein an outer surface of the cartridge is tangent to an inner surface of the shielding roller, and the plurality of radiation sources are disposed on a side, tangent to the shielding roller, in the cartridge.

8. The focusing head according to claim 7, wherein the shielding roller comprises a roller body, and a plurality of ray channels disposed on a side wall of the roller body; and
wherein the plurality of radiation sources are communicated with the plurality of ray channels in one-to-one correspondence when the focusing head is in a source-on state, and the plurality of radiation sources are staggered with the plurality of ray channels when the focusing head is in a source-off state.

9. The focusing head according to claim 8, wherein the first driving assembly is configured to drive the cartridge to rotate when the focusing head is switched between the source-on state and the source-off state.

10. The focusing head according to claim 8, wherein the second driving assembly is configured to drive the shielding roller to rotate when the focusing head is switched between the source-on state and the source-off state.

11. The focusing head according to claim 8, wherein when the focusing head is switched between the source-on state and the source-off state, the first driving assembly is configured to drive the cartridge to rotate in a first direction and the second driving assembly is configured to drive the shielding roller to rotate in a second direction, wherein the first direction is opposite to the second direction.

12. The focusing head according to claim 10, wherein the cartridge and the shielding roller rotate relative to each other at an acute angle.

13. The focusing head according to claim 11, wherein the first driving assembly is configured to drive the cartridge to rotate by 180°, and the second driving assembly is configured to drive the shielding roller to rotate by 90°.

14. The focusing head according to claim 8, wherein an external diameter of the cartridge is less than an internal diameter of the shielding roller.

15. The focusing head according to claim 14, wherein the cartridge is driven by the first driving assembly to rotate around a center axis of the shielding roller, and the shielding roller is driven by the second driving assembly to rotate around the center axis of the shielding roller.

16. The focusing head according to claim 15, wherein a distance between a center axis of the cartridge and the center axis of the shielding roller is 22 mm.

17. The focusing head according to claim 1, further comprising: a shielding layer for cladding the shielding roller.

18. The focusing head according to claim 8, further comprising: a collimator with a plurality of collimation channels thereon, wherein the plurality of ray channels are communicated with the plurality of collimation channels in one-to-one correspondence when the focusing head is in the source-on state.

19. The focusing head according to claim 1, wherein a material of the shielding roller comprises a tungsten alloy.

20. Radiotherapy equipment, comprising a rotating gantry and a focusing head connected to the rotating gantry, wherein the focusing head comprises:
a cartridge configured to carry a plurality of radiation sources;
a shielding roller with the cartridge therein;
a first driving assembly connected to the cartridge and configured to drive the cartridge to rotate; and
a second driving assembly connected to the shielding roller and configured to drive the shielding roller to rotate.
